(19) United States
(12) Patent Application Publication
(10) Pub. No.: US 2003/0153741 A1
(43) Pub. Date: Aug. 14, 2003

(54) Methods for separating oligonucleotides

(76) Inventor: Marek Kwiatkowski, Uppsala (SE)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/071,585

(22) Filed: Feb. 8, 2002

(51) Int. Cl. C12Q 1/68; C07H 21/04

(52) U.S. Cl. 536/25.4; 435/6

(57) ABSTRACT

Methods for separating oligonucleotides are described. The oligonucleotides to be separated include separation tags attached to each end of the oligonucleotides.

### TECHNICAL FIELD

This invention relates to purifying organic chemicals, and more particularly to purifying chemically synthesized oligonucleotides.

### BACKGROUND

During chemical synthesis of oligonucleotides, failed couplings and/or side reactions can take place that produce non-full-length or incomplete oligonucleotides. In addition, acid-catalyzed depurination can occur, resulting in cleavage of the oligonucleotide backbone during oligonucleotide deprotection. As a consequence, a population of oligonucleotides is produced during chemical synthesis of oligonucleotides, which must be purified to obtain the desired oligonucleotide.

### SUMMARY

The invention is based on methods for purifying oligonucleotides using separation tags attached to the 3′ and 5′-ends of oligonucleotides. In one aspect, the invention features a method for separating oligonucleotides. The method includes a) providing a plurality of oligonucleotides, the plurality of oligonucleotides including at least one bifunctional oligonucleotide and at least one non-bifunctional oligonucleotide (e.g., depurinated or truncated oligonucleotides), wherein each at least one bifunctional oligonucleotide includes a first separation tag attached to a first end of the at least one bifunctional oligonucleotide and a second separation tag attached to a second end of the at least one bifunctional oligonucleotide, and wherein cleavage of the first or the second separation tags yields an oligonucleotide having a 3′hydroxyl moiety; b) contacting the plurality of oligonucleotides with a separation medium under conditions effective for adhering the at least one bifunctional oligonucleotide to the separation medium; and c) selectively eluting at least one non-bifunctional oligonucleotide. The method further can include eluting the at least one bifunctional oligonucleotide. The method also can include cleaving either the first separation tag or the second separation tag; and eluting an oligonucleotide lacking the non-cleaved separation tag.

The first or the second separation tags can interact with the separation medium via a noncovalent interaction (e.g., hydrophobic, hydrophilic, hydrogen bonding, metal-complexing, ionic, or antigen-antibody interactions) or via a covalent bond (e.g., disulfide, hydrazo, alkoxyamino, or reactive carbonyl bonds). The first and second separation tags can be different. The first or second separation tags can include a separation unit selected from the group consisting of alkoxytrityl, alkoxypixyl, alkyldithioformacetal, methylthioalkyl, derivatives of mercaptodimethoxytrityl or mercaptotrityl, and a hydrocarbon chain introduced in a form of a linear or branched diol, and combinations thereof. The alkoxytrityl can be selected from the group consisting of 4-decyloxymethoxy trityl (C10Tr), 4-hexyloxymethoxytrityl (C6Tr), dimethoxytrityl (DMTr), and monomethoxytrityl (MMTr). The alkoxypixyl can be 4-octadecyloxyphynylxanthyl (C18-Px). The separation unit can be a derivative of a mercaptodimethoxytrityl or mercaptotrityl. The separation unit can be a methylthioalkyl moiety. The separation unit can be a hydrocarbon chain introduced in a form of a linear or branched diol.

A cleavable unit of either of the first or second separation tags can be selected from the group consisting of acid labile, fluoride ion labile, photolabile, redox labile, and electrophile labile moieties. The redox labile moiety can include a dithioformacetal moiety. The cleavable unit of either of the first or second separation tags can include a siloxyl or disyloxyl moiety. The cleavable unit of either of the first or the second separation tags can include an alkylthiomethyl moiety or a hydrocarbyldithiomethyl moiety.

The separation medium can be selected from the group consisting of affinity, hydrophobic interaction, hydrophilic interaction, metal-chelating, ion exchange, covalent coupling, and antigen-antibody affinity separation media. In particular, the separation medium can be an ion exchange separation medium, a reversed phase separation medium, or a mixed-mode type separation medium. The mixed-mode type separation medium can include a reversed phase and ion exchange separation media or a covalent coupling separation medium (e.g., a separation medium based on the formation of a disulfide bond). The separation medium can include a first separation medium and a second separation medium, the first separation medium effective for adhering to the first separation tag and the second separation medium effective for adhering to the second separation tag.

In another aspect, the invention features a method for separating oligonucleotides. The method includes a) providing a plurality of oligonucleotides, wherein the plurality of oligonucleotides includes at least one bifunctional oligonucleotide and at least one non-bifunctional oligonucleotide, and wherein each at least one bifunctional oligonucleotide includes a first separation tag attached to a first end of the at least one bifunctional oligonucleotide and a second separation tag attached to a second end of the at least one bifunctional oligonucleotide, and wherein cleavage of the first or the second separation tags yields an oligonucleotide having a 3′ hydroxyl moiety; b) contacting the plurality of oligonucleotides with a separation medium under conditions effective for adhering the at least one bifunctional oligonucleotide to the separation medium; c) eluting non-bifunctional oligonucleotides lacking the first separation tag without eluting the bifunctional oligonucleotides; d) cleaving the first separation tag from the oligonucleotides retained on the separation medium; and e) eluting non-bifunctional oligonucleotides lacking the second separation function. The cleaving step can be facilitated using TBAF or an acid.

In yet another aspect, the invention features a composition that includes a plurality of oligonucleotides, each oligonucleotide including a first separation tag attached to a first end of the oligonucleotide and a second separation tag attached to a second end of the oligonucleotide, wherein cleavage of the first or the separation tags yields an oligonucleotide having a 3′ hydroxyl moiety; and b) a separation medium, the plurality of oligonucleotides adhering to the separation medium. The separation medium can include a first separation medium and a second separation medium, the first separation medium and the second separation medium being different separation media.

Unless otherwise defined, all technical and scientific terms and abbreviations used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features and advantages of the invention will be apparent from the following detailed description, and from the claims.

### DESCRIPTION OF DRAWINGS

FIG. 1 is a chart describing the separation characteristics for a plurality of oligonucleotides containing a variety of separation functions at their 3′ and 5′ ends.

FIG. 2A and FIG. 2B are chromatograms illustrating the elution profiles for separating oligonucleotides.

### DETAILED DESCRIPTION

In general, methods of the invention allow a plurality of chemically-synthesized oligonucleotides, e.g., oligonucleotides synthesized on a solid support, to be separated based on separation tags attached to first and second ends of the oligonucleotides. The term “oligonucleotide” includes oligomers of ribonucleotides and deoxyribonucleotides that have a 3′-5′ phosphodiester backbone, as well as oligomers of ribonucleotides and deoxyribonucleotides with backbone structures differing from the standard 3′-5′ phosphodiester linkage (e.g., peptide-nucleic acids (PNAs), methyl phosphonate, or phosphorothioate linkages). The term “oligonucleotide” also includes oligomers that contain non-standard base moieties such as inosine or nubularine, modified base moieties, modified sugar moieties, and combinations of such moieties. For example, the nitrogenous bases or sugar moieties can be modified to include reactive functionality (e.g., C5 propyne, halide, or biotin) and labels (e.g. radioactive, luminescent, electroluminescent, visible, near-IR, and fluorescent). Methods for synthesizing oligonucleotides, including oligonucleotides containing non-standard bases, are known in the art. For example, oligonucleotides can be assembled by the β cyanoethyl phosphoramidite method. See, for example, “Oligonucleotide Synthesis: A Practical Approach,” ed. M. J. Gait, IRL Press, 1984, WO92/09615; and WO98/08857 for a description of oligonucleotide synthesis methods. Examples of solid phase syntheses of PNAs using disulfide anchoring linkers can be found in Aldrian Herrada, G. et al. J. Peptide Sci. 4, 266-281 (1998). Automated oligonucleotide synthesizer machines can be used to produce oligonucleotides. Such synthesizers are known and are available from a variety of companies including Applied Biosystems and Amersham Pharmacia Biotech.

Oligonucleotides having separation tags on first and second ends are designated “bifunctional” in the present application. It should be noted that bifunctional oligonucleotides includes full-length oligonucleotides, i.e., oligonucleotides of the desired length and containing both separation tags, as well as oligonucleotides that contain both separation tags, but are less than the desired length, e.g., due to an internal deletion. “Non-bifunctional” refers to any oligonucleotide that does not contain both separation tags.

Separation Tag

The term “separation tag” refers to chemical groups or moieties bonded to either the 3′ or 5′ end of an oligonucleotide that allows oligonucleotides having the separation tag to be separated from other oligonucleotides that lack this function. Useful separation tags are selectively cleavable, either chemically or photochemically, such that the separation tag can be removed from each end of the oligonucleotide. Thus, a separation tag includes a “separation unit” and a “cleavable unit,” which can be the same or different chemical moieties. Separation tags can be attached to an oligonucleotide during chemical synthesis, e.g., as a part of a linker that attaches the oligonucleotide to the solid support. Alternatively, a separation tag can be added before or after the synthesis using methods known to those of ordinary skill in the art to which this invention pertains.

Separation tags are located at the ends of the oligonucleotides, i.e. located before the first nucleotide of the oligonucleotide and after the last nucleotide of the oligonucleotide. For example, separation tags attached to the ends of an oligonucleotide having 30 nucleotides, i.e., a 30 mer, are located before nucleotide 1 and after nucleotide 30. It should be noted that first and second ends do not refer to a particular or specific end of the oligonucleotide. Rather, first end and second end simply demonstrate that there are two ends to each oligonucleotide.

Separation tags on first and second ends of the oligonucleotide can be the same or different. For example, the separation tags can be hydrophobic on each end, or a hydrophobic function can be on one end while a covalent function can be on the other end.

Synthesis of Bifunctional Oligonucleotides

Oligonucleotide chemical syntheses typically utilize a solid support to which one or more protected nucleotides are attached via a linker to the nucleotide's 3′-oxygen. As additional nucleotide monomers are added successively, the resulting oligonucleotide is extended in a 3′ to 5′-end direction. After an oligonucleotide chain of the desired length is obtained, the oligonucleotide is cleaved from the solid support and the protecting groups are removed. A linker refers to any molecule containing a chain of atoms, e.g., carbon, nitrogen, oxygen, etc., that serves to link the molecules to be synthesized on the support with the support. The linker is usually attached to the support via a covalent bond before synthesis on the support starts, and provides one or more sites for attachment of precursors of the molecules to be synthesized. It is to be understood that, at times, linkers include one or more nucleotides, e.g., polyT, that are not part of the finished full-length oligonucleotide. Nucleotides that are part of the linker, but are not part of the finished full-length oligonucleotide, are not considered to be the 3′-end of an oligonucleotide. Typically, the linker is base labile so that oligonucleotides may be cleaved from the solid-support using, for example, ammonia. A non-limiting example of a base labile linker is succinyl alkyl amine.

Separation tags on the 3′ end of the oligonucleotide can be a component of the linker between the solid support and the first nucleotide, i.e., 3′-end, of the oligonucleotide. The separation tag on the 3′ end of the oligonucleotide typically is stable under treatment in aqueous ammonia so the separation tag will not be cleaved from the oligonucleotide when the oligonucleotide is released from the solid support. The separation unit of a separation tag can interact with separation media based on one or more chemical interactions, including, for example, hydrophobic, hydrophilic, ionic, hydrogen bonding, and/or covalent interactions. For example, interactions between a separation unit and a separation medium include ion exchange (e.g., anion or cation exchange), hydrophobic (e.g., reversed phase), metal-chelating (e.g., Ni (+2) complexing/histidine tagging), protein interactions (e.g., enzyme/substrate), hapten/antigen/antibody, and/or covalent (e.g., thiol/disulfide, hydrazo, alkoxyamino, or reactive carbonyl). In particular, linear or branched diols such as 1,10-decanediol or other hydrophobic diols can be used as starting materials for preparation of hydrophobic separation units of a separation tag.

A cleavable unit of the separation tag on the 3′ end of the oligonucleotide can be attached to the 3′ oxygen of the first nucleotide in the oligomer. Suitable cleavable units on the 3′ end of the oligonucleotide regenerate a free 3′ OH after being cleaved. Disiloxyl groups (“DSi”), alkyl thiomethyl and hydrocarbyldithiomethyl groups, photolabile groups (e.g., o-nitrobenzyl groups), redox active groups (e.g., disulfide-containing or hydrocarbyldithiomethyl groups), and electrophilic reagents (e.g., alkyl thiomethoxy groups) are examples of suitable cleavable units that can be components of a linker. A disiloxyl-containing linker can be created using the methods provided in Kwiatkowski et al., Nucleic Acids Res., 24:4632-4638 (1996); Kwiatkowski et al., Nucleic Acids Res., 27(24):4710-14 (1999); or WO 98/08857. Photolabile functions can be introduced into linkers using the methods of Greenberg, Tetrahedron Lett. 34:251-254 (1993). Hydrocarbyldithiomethyl group-containing linkers can be introduced using the methods provided in U.S. Pat. No. 6,309,836. In particular, a 3′-methylthioalkyl phosphoramidite can be prepared from 5′ dimethoxytrityl, 3′-methylthiomethyl thymidine according to the methods of Veeneman et al. Tetrahedron, 47:1547-1562 (1991). The methods of Ducharme & Harrison, Tetrahedron Lett. 36:6643-6646 (1995) can be used to convert 5′ dimethoxytrityl, 3′-methylthiomethyl thymidine to 5′-dimethoxytrityl, 3′-methylthiobutanol thymidine, which can then be phosphitylated according to well-known procedures to prepare a 3′-methylthioalkyl phosphoramidite.

The above-discussed groups also can be used as separation tags on the 5′-end of an oligonucleotide. A 5′-end separation tag can be introduced together with the 5′ terminal nucleotide as this terminal, appropriately derivatized phosphoramidite is added to the oligomer during synthesis. Suitable separation tags for the 5′ end of the oligonucleotide include those that provide a separation function, in addition to protecting the terminal hydroxyl residue, and may be cleaved selectively from the synthesized oligomer. The terminal nucleotide may include a sugar and/or base —OH or —NH₂ protecting group that also serves as a separation function. Additionally, several useful functional groups can be incorporated at the 5′-end of the oligonucleotides. These functional groups includes appropriately derivatized phosphate, thiol, amine, hydrazide, alkoxyamine or biotin, which can be prepared by known methods. For example, a thiol or phosphate can be added via the trityl-S procedure of Connolly, Tetrahedron Letters, 28(4):463-66 (1987). Hydrocarbyldithiomethyl groups can be added to hydroxyls using the methods described in U.S. Pat. No. 6,309,836.

Useful 5′ separation functions include well-known hydrophobic groups such as dimethoxytrityl (“DMTr”), pixyl, alkoxytrityl, and alkoxypixyl protecting groups. For example, octadecyloxypixyl phynylxanthyl (“C₁₈Px”), 4-decyloxymethoxy trityl (“C₁₀Tr”), 4-hexyloxymethoxytrityl (“C₆Tr”), and monomethoxytrityl (MMTr), a hydrocarbon chain introduced in a form of a linear or branched diol, alkyldithioformacetal, methylthioalkyl and derivatives of mercaptodimethoxytrityl or mercaptotrityl can be used as separation functions. The 5′ separation tag also can be added in a separate reaction. Different types of substituted trityl groups can be introduced on the 5′ position in the synthetic oligonucleotides in a process of trityl exchange as described in U.S. Pat. No. 5,319,079.

By way of example, bifunctional oligonucleotides can be produced using one or more of the following steps. A 3′-end separation tag can be attached onto a solid support through a linker cleavable in aqueous ammonia (e.g., by incorporation of a simple ester moiety). Briefly, a linker can be attached to a solid support, such as a 1000 Å controlled pore glass (CPG), that has been previously derivatized with one or more of the standard nucleotides by a single or repeated introduction of phosphoramidite derivatives of sufficiently hydrophobic linear or branched diols. An appropriately protected nucleoside can be silylated using 1,3-dichloro-1,1,3,3-tetraisopropyl disiloxane in dry pyridine and imidazole followed by a reaction with a selected diol (e.g. tetraethylene glycol). The terminal hydroxyl group of the silylated nucleoside then can be converted to a phosphoramidite and added to the free hydroxyl group at the end of the linker. Coupling of the phosphoramidite derivative to the free end of the linker introduces the starting 3′ nucleoside of the oligonucleotide. Additional nucleoside phosphoramidites are coupled sequentially to generate the oligonucleotide of the desired length using known oligonucleotide synthesis methods. Finally, a 5′-end separation tag can be introduced onto the full-length oligonucleotide by coupling a suitably derivatized nucleoside phosphoramidite at the 5′ terminus of the growing oligonucleotide chain. After deprotecting and cleaving the oligonucleotides from the solid support using concentrated ammonia, a bifunctional oligonucleotide of Structure 1 can be obtained.

### Structure 1

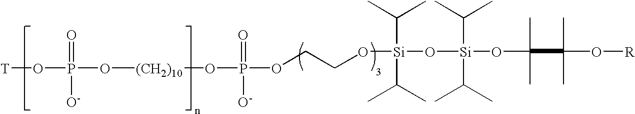

Referring to Structure 1 above, “T” represents one or more nucleotides or non-nucleotide equivalents remaining on the 3′-end of the bifunctional oligonucleotide after the completed oligonucleotide is separated from the solid support. For example, in Structure 1, “T” represents one or more thymidine nucleotides.

The variable “n” can be 0, 1, or 2, and represents the number of separation units in the separation tag added to a CPG oligonucleotide solid support. A variety of separation units can be used in the separation tag of the invention. In the embodiment depicted in Structure 1, the variable “n” represents, for example, the number of phospho-1,10-decanediol units. Increasing the number of 1,10-decanediol units increases the hydrophobicity of the 3′-end separation function. In this embodiment, the overall 3′-end linker includes the one or more thymidine nucleotides, phospho-1,10-decanediol units, phospho-triethylene glycol unit, and disiloxyl components (i.e., the cleavable unit of the separation tag). The “o-|—|-o” represents the oligonucleotide. “R” represents a 5′-end separation tag linked to the 5′-OH of the oligonucleotide.

In some embodiments, a 3′-methylthioalkyl phosphoramidite is substituted for the disiloxyl amidite discussed above. The 3′-methylthioalkyl phosphoramidite may then be coupled to the free hydroxyl group of the 1,10-decanediol unit. After completion of the oligonucleotide synthesis cycle and standard aqueous ammonia deprotection, the synthesized bifunctional oligonucleotides have the following Structure 2.

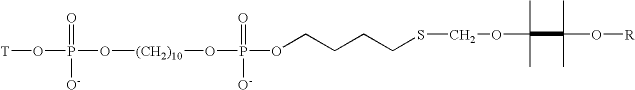

### Structure 2

In some embodiments, a disiloxyl linkage can be added to a solid support, prepared by coupling of 1-O-dimethoxytrityl-hexyl-disulfide, 1′-[(2-cyanoethyl)-(N,N-diisopropyl)]-phosphoramidite to the appropriately derivatized solid support. Dithiothreitol can be used to reduce the disulfide linkage thereby creating an oligonucleotide that can be covalently bound to a separation medium containing thiol reactive functions. After reduction, an oligonucleotide synthesized on the disiloxyl-containing support can have Structure 3.

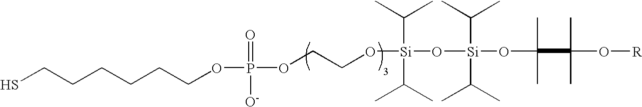

### Structure 3

Separation Media

The separation tags described above determine the type of separation medium that can be used to isolate the bifunctional oligonucleotides. Thus, any known separation medium appropriate for the particular separation unit can be used to separate bifunctional and non-bifunctional oligonucleotides, and obtain purified bifunctional oligonucleotides. Useful separation media contain chemical groups or moieties that interact with a separation unit based on one or more chemical interactions such as a hydrophobic, hydrophilic, ionic, and/or covalent interactions. For example, separation media including groups that can interact with a separation unit via ion exchange (e.g., anion or cation exchange), hydrophobic (e.g., reversed phase), metal-complexing (e.g., Ni (+2) complexing histidine tagging), affinity interactions, hapten/antigen/antibody, and/or covalent (e.g., thiol/disulfide) type interactions are useful.

The types of separation media that may be used are not limited, and includes, for example, derivatized silica, resins, appropriately modified membranes or particle loaded membranes, and other supports. Suitable separation media can be obtained commercially from, for example, Amersham Pharmacia Biotech (Piscatawy, N.J.); Amicon, Inc. (Beverly, Mass., now owned by Millipore); EM Separations Technology (Gibbstown N.J.); Vydac (Hesperia, Calif.); and/or Bio-Rad Laboratories (Hercules, Calif.). Non-limiting examples of separation media include hydrophobic media such as silica-based C4, C8, and C18 supports or non-silica-based materials such as butyl sepharose, octyl sepharose, and phenyl sepharose. Ion-exchange materials (especially anion exchange materials) include Mini Q, Mono Q, Q sepharose, SAX trimethyl aminopropyl, DEAE sepharose, and EMD DEAE-650. Affinity separation materials are represented by pairs of, for example, immobilized streptavidin and iminobiotin-labeled oligonucleotides, immobilized nitrostreptoavidin and biotin-labeled oligonucleotides (Morag, et al. Anal. Biochem., 243:257-263) or immobilized salicylhydroxyamic acid and phenylboronic acid labeled oligonucleotides (Bergseid et al., BioTechniques, 29(5):1126-1133). Separations media relying on formation of a covalent bond can be exemplified by pyridinedithiopropyl-sepharose and thiol-labeled oligonucleotides, immobilized hydroxylamine or hydrazide like agarose adipic acid hydrazide and aldehyde labeled oligonucleotides. Methods and recommended conditions for using the exemplified resins and membranes are well known and readily available to the skilled artisan.

Systems for separating oligonucleotides can include one or more HPLC columns and/or separation cartridges. Systems containing a variety of elements (e.g., valves, pumps, injection units and fraction collectors) and columns or cartridges can be configured to automate the separation of bifunctional oligonucleotides. Columns or cartridges can be run in sequential or parallel fashion depending on the isolation order preferred for particular oligonucleotides.

Bifunctional oligonucleotides can be separated from non-full length oligonucleotides using a single separation media or multiple types of separation media (i.e., mixed mode), depending on the nature of the first and second separation tags. In some embodiments, a bifunctional oligonucleotide can be separated from non-bifunctional oligonucleotides using a single column and, in some instances, in a single run. For example, bifunctional oligonucleotides containing first and second separation tags that are hydrophobic can be separated from non-bifunctional oligonucleotides using one or more types of reversed phase separation media as follows. A mixture of bifunctional and non-bifunctional oligonucleotides can be contacted with an appropriate separation medium that retains bifunctional oligonucleotides as well as non-bifunctional oligonucleotides containing the separation tag that interacts more strongly with the separation medium. The retained oligonucleotides then are eluted from the column and the more strongly interacting separation tag is cleaved. The resulting mixture of oligonucleotides can be contacted with a separation medium and the desired oligonucleotide can be obtained. It is to be understood that a separation unit may be cleaved from a bifunctional oligonucleotide while adhering to a separation medium, i.e., without first eluting the bifunctional oligonucleotide. Similarly, a separating tag may be cleaved from an oligonucleotide possessing only one separation tag while adhering to the separation medium.

Separation of oligonucleotide mixtures may be performed using separation medias in a column (i.e., mixed-mode). For example, one region of a column containing a separation medium can interact with a first separation tag, while a different separation medium in a different region of the same column can interact with a second separation tag. Alternatively, the different separation media are interspersed in the column.

After purification, oligonucleotide concentration can be determined by UV absorbance (λmax 260 nm). Quantitative and qualitative comparison of components in the final purified product can be obtained directly from a liquid chromatography instrument equipped with a UV detector. For example, peak areas can be calculated directly from an HPLC chromatogram for all chromophore containing components. A ratio of the integrated peak areas is used to calculate the purity of the desired product.

The invention will be further described in the following examples, which do not limit the scope of the invention described in the claims.

### EXAMPLES

Reagents and Analytic and Preparative Methods Used in the Examples

Unless indicated otherwise, the following reagents and methods were used in the Examples that follow this methods section.

5′-O-(4,4′-dimethoxy)trityl thymidylyl 3′-O-(1,1,3,3-tetraisopropyl-disiloxyl-3)-(1-O-3,6,9-trioxa)undecan-11-ol and its phosphoramidite derivative were prepared according to PCT Publication No. WO 98/08857. Commercially available CPG (1000 Å; CPG Inc., Fairfield; or Applied Biosystems, Foster City, Calif.) was thiopropylsilanized with methods similar to those described by Pon, R T, “Chapter 19 Solid-phase Supports for Oligonucleotide Synthesis,” Methods in Molecular Biology Vol. 20 Protocols for Oligonucleotides and Analogs, 465-497, Ed. S. Agrawal, Humana Press Inc., Towata, N.J. (1993). Dimethoxytrityl-hexyl-disulfide,1′-[(2-cyanoethyl)-(N,N-diisopropyl)]-phosphoramidite was obtained from Glen Research, Sterling, Va. All commercial chemicals were of synthesis quality and were used without further purification.

Oligonucleotide syntheses were performed on an ABI 394 DNA Synthesizer. All couplings were performed using amidites protected by benzoyl (dA, dC) and/or isobutyryl (dG) groups at the exocyclic amine functions, under conditions recommended by the manufacturer for 0.2 μmol scale synthesis. If demanded, the last nucleoside phosphoramidite was substituted with a nucleoside phosphoramidite derivatized with another 5′protecting group, or the final dimethoxytrityl (DMTr) groups were exchanged by other trityl derivatives according to the trityl exchange method (see U.S. Pat. No. 5,319,079).

Analytical liquid chromatography of ammonia deprotected oligonucleotides was performed on a Hitachi-Merck La Chrom HPLC system, equipped with a LiChrosorb RP 18 (5 μm) column, diode array detector, using a 40 minutes linear gradient of solvent A:acetonitrile (“MeCN”) 5% v/v in 0.1 M triethylammonium acetate (“TEAA”), pH 7.0, and solvent B:acetonitrile 80% v/v in 0.1 M TEAA, pH 7.0.

To remove the disiloxyl groups, 0.5 M tetrabutylammonium fluoride (“TBAF”) in dry tetrahydrofuran (“THF”) (200 μl) was added to the partially evaporated oligonucleotide solution, where water forms only a thin film on the walls of a round-bottomed 10 ml flask. The mixture was incubated for 2 h at 20° C. Alternatively, the disiloxyl linker can be cleaved using 200 μl 0.5 M TBAF in dry DMF at 65° C. for 30 min. Trityl and pixyl-type groups were removed by exposing the full-length oligonucleotides with an 80% aqueous acetic acid solution for 20 min at 20° C. followed by evaporating the acid.

Isolated and fully deprotected oligonucleotides were analyzed on an analytical ion-exchange Mini Q HPLC column, using a denaturing (pH 12) salt gradient elution profile consisting of solvent A (0.01 M NaOH), and solvent B (0.01 M NaOH+1.0 M NaCl) run from 0% to 100% B in 40 min. Oligonucleotides longer than 25 bases were analyzed by capillary electrophoresis on a Beckman MDQ system using a ssDNA 100-R capillary kit (capillary length 30 cm).

### Example 1

Chemical Synthesis and Single HPLC Run Separation of Bifunctional Oligonucleotides Containing First and Second Separation Tags from Non-Bifunctional Oligonucleotides when the First and Second Separation Tags are Removable Under Different Conditions.

Oligonucleotides containing 25 nucleotides (25-mer), consisting solely of guanine and adenine, i.e., G(AG)₁₂, were synthesized according to the methods described above. Oligonucleotides were synthesized on a CPG oligonucleotide solid support purchased from Applied Biosystems, Foster City, Calif. The CPG solid support was available preloaded with any of the standard deoxynucleosides. The 25-mers were synthesized according to the general structure indicated below, which represents an oligonucleotide after partial deprotection and removal from the CPG solid support.

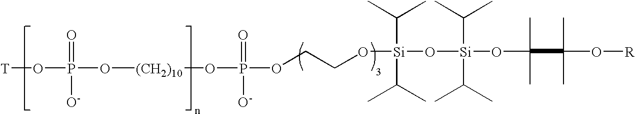

Referring to the general structure above, “T” represents a single thymidine unit bound on the commercial CPG oligonucleotide solid support. The variable “n” was 0, 1, or 2 and represents the number of phospho-1,10-decanediol units added to the CPG oligonucleotide solid support. The separation tag attached to the 3′ end of the oligonucleotide (3′ separation tag) includes the phospho-1,10-decanediol units, the phospho-triethylene glycol unit, and the disiloxyl linker (DSi). Three different variations of the 3′ separation tag were prepared, in which n=0, 1, or 2, referred to as DSi, DSiC10, and DsiC20, respectively. The “o-|—|-o” represents the oligonucleotide. “R” represents a 5′-hydroxyl separation tag (5′separation tag) such as octadecyloxypixyl (C₁₈Px), 4-decyloxymethoxy trityl (C₁₀Tr), 4-hexyloxymethoxytrityl (C₆Tr), or dimethoxytrityl (DMTr). The 5′ separation tag introduced hydrophobic properties to the 5′ end of the oligonucleotide.

The synthesized oligonucleotides received a variety of 3′ and 5′ separation tags according to the nine systems in Table 1. Altering the individual 3′ separation tags or 5′ separation tag altered the hydrophobic properties of both full-length oligonucleotides and any fragments containing either a 3′ or 5′ separation tag.

TABLE 1

| | 5′ Separation | 3′ Separation | |
|---|---|---|---|
| System | Tag | Tag | n |
| 1 | DMTr | Dsi | 0 |
| 2 | C₆Tr | Dsi | 0 |
| 3 | C₁₀Tr | Dsi | 0 |
| 4 | DMTr | DsiC₁₀ | 1 |
| 5 | C₆Tr | DsiC₁₀ | 1 |
| 6 | C₁₀Tr | DsiC₁₀ | 1 |
| 7 | DMTr | DsiC₂₀ | 2 |
| 8 | C₆Tr | DsiC₂₀ | 2 |
| 9 | C₁₀Tr | DsiC₂₀ | 2 |

Chemically synthesized oligonucleotides were deprotected and removed from the CPG solid support by contacting the solid support with concentrated aqueous ammonia and placing the solid support in an oven at 65° C. for 12 hours. Ammonia treatment removed base protecting groups, phospate protecting groups, and cleaved the ester group linking the oligonucleotides to the solid support. Oligonucleotides containing apurinic sites generated during the oligonucleotide synthesis were simultaneously cleaved via a process of base-catalyzed cleavage of all apurinic sites. The 3′-disiloxyl separation tag and 5′-acid labile seperation tag remained intact. The deprotected oligonucleotides were partially concentrated and desalted on a NAP 10 Sephadex column. The concentrated and desalted deprotected oligonucleotides were analyzed by HPLC using a 5 μm Lichrosorb RP18 column according to the method above.

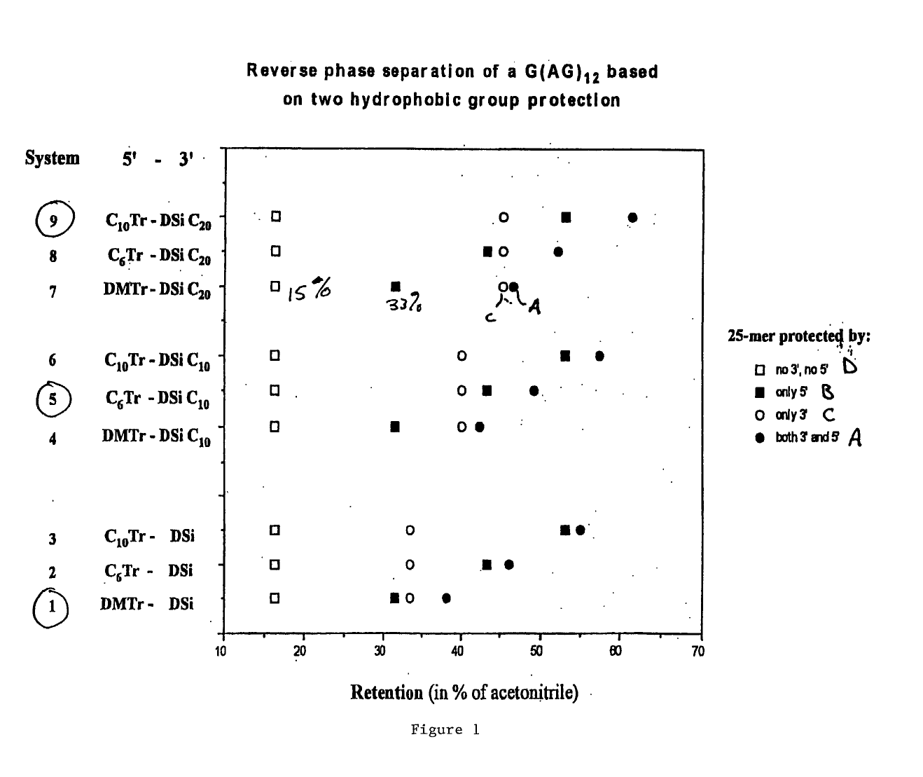

FIG. 1 shows the RP18 retention properties, i.e., approximate percentage acetonitrile used for elution, for various 25-mers including full-length and non-bifunctional oligonucleotides were analyzed according to systems 1-9 of Table 1.

Oligonucleotides containing a C₁₈Px 5′ separation tag also were prepared, but are not represented in FIG. 1, since the C₁₈Px 5′ separation tag containing species demanded stronger elution conditions outside of the range shown in FIG. 1. For ease of reference, each oligonucleotides species represented in FIG. 1 was placed in one of the following four categories: Group A) Bifunctional oligonucleotides having both 3′ and 5′ separation tags indicated in Table 1, solid circles; Group B) Non-bifunctional oligonucleotides having only 5′ separation tags indicated in Table 1(e.g., 5′-end of depurinated and cleaved oligonucleotides), solid squares; Group C) Non-bifunctional oligonucleotides having only 3′ separation (e.g., prematurely truncated oligonucleotides and 3′-end of depurinated and cleaved oligonucleotides) tags indicated in Table 1, open circles; and Group D) Non-bifunctional oligonucleotides lacking both the 5′ and the 3′ separation tags (e.g., oligonucleotide fragments resulting from cleavage of at least two apurinic sites located within the same oligonucleotides chain) indicated in Table 1, open squares.

A mixture of oligonucleotides prepared according to systems 1, 5 and 9 were sufficiently separable using the acetonitrile elution profile to facilitate isolating full-length oligonucleotides, i.e., Group A, in a single chromatographic run. The 3′ and 5′ separation tags then were released from the full-length oligonucleotides using the following process. The isolated Group A fraction was dried and then resuspended in 80% aqueous acetic acid for 20 minutes to remove acid labile separation tag. Next, the full-length oligonucleotides were re-dried and then resuspended in 0.5 M TBAF for 2 hours to remove the siloxyl functional groups. The final product was desalted on a disposable gel filtration column e.g. NAP 5 (Pharmacia).

### Example 2

Separating Bifunctional Oligonucleotides from Non-Bifunctional Oligonucleotides Using Multiple HPLC Runs.

Oligonucleotides ranging in length from 20 to 105 nucleobases were prepared according to the methods of Example 1, using the 3′ and 5′ separation tag strategy of system 7 (Table 1). The reaction mixtures included full-length (Group A) and shorter side-product oligonucleotides representing Groups B, C, and D. The reaction mixtures were loaded onto and separated using a 5 μm RP18 column (as described above) operated in an isocratic mode (60% MeCN, 0.1M TEAA).

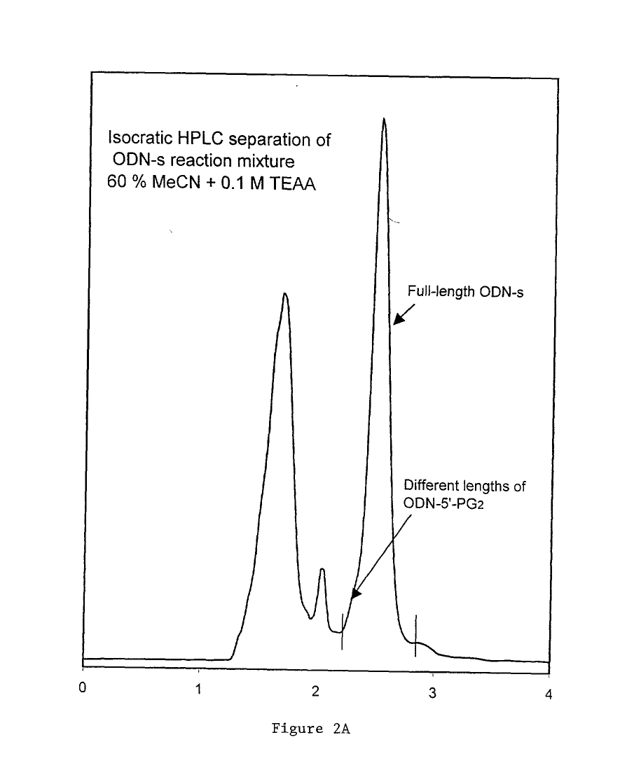

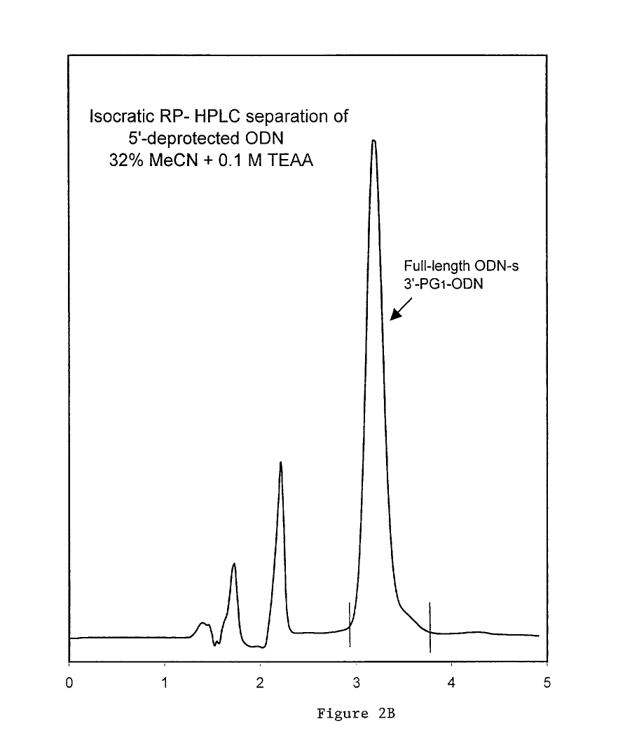

FIG. 2a illustrates aspects of the eluant profile. The hash marks in FIG. 2a show the starting and ending points of the eluant fraction collected. The collected eluant fraction contained only oligonucleotides belonging to the Groups A and C. The collected eluant fraction was concentrated and treated with 0.5 M TBAF in dry THF (200 μl) to remove the 3′-disiloxyl separation tag. Triethylamine (20 μl) was added to the concentrated eluant to prevent cleavage of the 5′-DMTr separation unit during the TBAF treatment. The 3′-separation tag-free oligonucleotide containing eluant was desalted after TBAF treatment and loaded onto the RP18 column equilibrated with 32% MeCN. FIG. 2b shows aspects of the 32% MeCN elution profile. The hash marks in FIG. 2b show the starting and ending points of the eluant fraction collected. The collected eluant fraction contained only full-length oligonucleotides having an intact 5′-separation tag. The collected eluant fraction was subsequently dried and detritylated using well-known methods.

The isocratic method for separating oligonucleotides was very fast, offering isolation of the desired oligonucleotides within 3 to 4 minutes. Changing the oligonucleotide length and/or sequence did not significantly affect the isolation times. An additional benefit of the isocratic method was that the column did not need to be reequilibrated between individual runs.

### Example 3

Separating Bifunctional Oligonucleotides from Non-Bifunctional Oligonucleotides by Mixed-Mode Separation.

A plurality of oligonucleotides including bifunctional and non-bifunctional oligonucleotides representing Groups A, B, C, and D in Example 1 and characterized by an intermediate hydrophobic 3′-separation tag (n=1), and a strongly hydrophobic C₁₈Px 5′-separation tag were separated using an anion exchange separation medium.

Oligonucleotide mixtures dissolved in water:ethanol (9:1) were loaded onto a Mono Q column equilibrated with water (Mono Q, Pharmacia Biotech, Uppsala Sweden). Mixtures of water and ethanol were used to prevent formation of the micelles by oligonucleotides that have a substantial detergent character. The loaded Mono Q column was washed with 3 column volumes of water. A NaCl gradient from 0 to 1 M NaCl in 15 minutes was applied to the column thereby removing Group D oligonucleotides. This was followed by a second MeCN gradient elution from 0 to 30% MeCN in 1M NaCl to elute Group C oligonucleotides. The Mono Q column was then equilibrated with water followed by 100% MeCN. Next, trichloracetic acid (“TCA”)(3%) in dichloromethane (“DCM”) was pumped through the column to cleave the 5′-end separation tag. The release of C₁₈Px separation tag was accompanied by the formation of a colored carbocation, which is visible to the naked eye. Accordingly, removal of the carbocation was easily monitored. The time necessary for the removal of the acid labile 5′ separation tag was less then 2 minutes.

The Mono Q column was washed with 100% MeCN and then with water. Another NaCl gradient from 0 to 1 M NaCl in 15 minutes was applied to the column thereby removing oligonucleotides of Group D formed from Group B during the TCA treatment. A second acetonitrile gradient from 0 to 30% MeCN in 1M NaCl was applied to the Mono Q column to elute the Group C oligonucleotides, which were formed by the depixylation of full-length material during the TCA treatment. The 3′-end disiloxyl separation tag was then removed from the collected material by treating the evaporated fraction with TBAF as described above for Example 1.

### Example 4

Separating Bifunctional Oligonucleotides from Non-Bifunctional Oligonucleotides Using a Mixed-Mode Separation.

5′-Dimethoxytrityl, 3′-methylthiomethyl thymidine was prepared according to Veeneman et al. Tetrahedron, 47:1547-1562 (1991). This material was converted to 5′-dimethoxytrityl, 3′-methylthiobutanol(1) thymidine in a reaction similar to that described by Ducharme & Harrison, Tetrahedron Lett. 36:6643-6646 (1995). The resulting thymidine derivative was phosphitylated according to the well-known procedures resulting in a 3′-methylthioalkyl phosphoramidite.

Oligonucleotide synthesis was performed as described in Example 3, with the exception that the disiloxyl amidite was substituted by the 3′-methylthioalkyl phosphoramidite. After a standard aqueous ammonia deprotection, the synthesized full-length oligonucleotides had the following general structure:

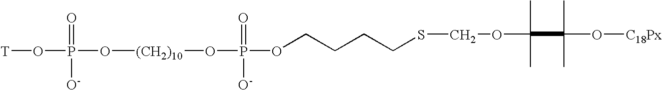

Bifunctional oligonucleotides were isolated using the method of Example 3, except that the second acetonitrile gradient elution step was replaced as follows. The Mono Q column was equilibrated with 0.1 M phosphate: 0.5 M NaCl buffer (pH 6.5). The 3′ separation tag was cleaved by passing a phosphate buffer: 0.1 M aqueous bromine (20:1 v/v) mixture through the column at a 1 ml/min rate. A 2 ml eluant was collected directly into a very diluted sodium bisulphate solution to quench any excess bromine. The 3′ and 5′ separation tag free oligonucleotides were obtained after a final desalting.

### Example 5

Separating Bifunctional Oligonucleotides from Non-Bifunctional Oligonucleotides Using a Mixed-Mode Separation.

1-O-dimethoxytrityl-hexyl-disulfide, 1′-[(2-cyanoethyl)-(N,N-diisopropyl)]-phosphoramidite (available from Glen Research, Sterling, Va.) was coupled to the support. A disiloxyl linkage, as shown in Example 1, was added by amidite incorporation. Forty two-nucleobase oligonucleotides were chemically synthesized on the disiloxyl-containing CPG solid support using standard conditions. The terminal 5′ position was capped with a C₁₈Px protected thymidine.

Oligonucleotides were released from the support by exposing the column to concentrated ammonia together with 25 mg of the reducing agent dithiothreitol. The mixture was placed in an oven at 55° C. for 12 hours to complete the oligonucleotide deprotection and disulfide cleavage.

The reduced and deprotected bifunctional oligonucleotides had the following general structure:

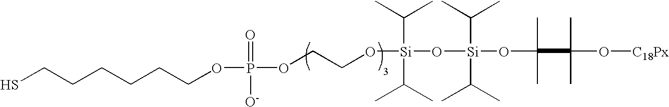

After partial concentration, the deprotected oligonucleotide mixture was desalted on an NAP 10 Sephadex column, which removed any excess DTT. The deprotected oligonucleotide mixture was applied at a rate of about 0.1 ml/min to a binary cartridge containing a reverse phase silica support (C-8) placed in the lower part of the cartridge, and a dithiopyridyl derivatized CPG support as the upper layer (28 μmol SS-Py/g). Oligonucleotides having the proper 3′ separation tag, in this case a free thiol, were covalently bound to the thiol reactive support via a disulfide bond. Oligonucleotides lacking a 3′-end free thiol bound to the lower hydrophobic support.

Oligonucleotides lacking a 3′ free thiol were washed away using a water:acetonitrile (4:6) mixture. The column then was equilibrated with water and washed with 10 μM DTT dissolved in 0.1 M TEAA (pH 7.0), which cleaved the disulfide bonds and released the oligonucleotides bound to the upper half of the binary cartridge. Under these conditions, the released oligonucleotides adhered to the lower hydrophobic support of the binary cartridge. The binary cartridge was washed with a 30% MeCN: 0.1M TEAA buffer, which eluted non-bifunctional oligonucleotide fragments from the cartridge. The desired oligonucleotides were eluted with a 70% MeCN: 0.1 TEAA buffer and collected. The collected fraction was evaporated, treated with 80% aqueous acetic acid for 20 minutes to remove the 5′ pixyl separation tag, evaporated again, and treated by TBAF to remove the disiloxyl linkage tag as described above. TBAF was removed using a disposable desalting column.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for separating oligonucleotides, said method comprising:
a) providing a plurality of oligonucleotides, said plurality of oligonucleotides comprising at least one bifunctional oligonucleotide and at least one non-bifunctional oligonucleotide, wherein each said at least one bifunctional oligonucleotide comprises a first separation tag attached to a first end of said at least one bifunctional oligonucleotide and a second separation tag attached to a second end of said at least one bifunctional oligonucleotide, and wherein cleavage of said first or said second separation tags yields an oligonucleotide having a 3′hydroxyl moiety;
b) contacting said plurality of oligonucleotides with a separation medium under conditions effective for adhering said at least one bifunctional oligonucleotide to said separation medium; and
c) selectively eluting at least one non-bifunctional oligonucleotide.

2. The method of claim 1, wherein said non-bifunctional oligonucleotides comprise depurinated or truncated oligonucleotides.

3. The method of claim 1, wherein either of said first or said second separation tags interacts with said separation medium via a noncovalent interaction.

4. The method of claim 1, wherein either of said first or said second separation tags interacts with said separation medium via a covalent bond.

5. The method of claim 4, wherein said covalent bond is selected from the group consisting of disulfide, hydrazo, alkoxyamino, and reactive carbonyl bonds.

6. The method of claim 3, wherein said noncovalent interaction is selected from the group consisting of hydrophobic, hydrophilic, hydrogen bonding, metal-complexing, ionic, and antigen-antibody interactions.

7. The method of claim 1, wherein said first and said second separation tags are different.

8. The method of claim 1, wherein either of said first or said second separation tags comprise a separation unit selected from the group consisting of alkoxytrityl, alkoxypixyl, alkyldithioformacetal, methylthioalkyl, derivatives of mercaptodimethoxytrityl or mercaptotrityl, and a hydrocarbon chain introduced in a form of a linear or branched diol, and combinations thereof.

9. The method of claim 8, wherein said alkoxytrityl is selected from the group consisting of 4-decyloxymethoxy trityl (C₁₀Tr), 4-hexyloxymethoxytrityl (C₆Tr), dimethoxytrityl (DMTr), and monomethoxytrityl (MMTr).

10. The method of claim 8, wherein said alkoxypixyl comprises 4-octadecyloxyphynylxanthyl (C₁₈-Px).

11. The method of claim 8, wherein said separation unit comprises a derivative of a mercaptodimethoxytrityl or mercaptotrityl.

12. The method of claim 8, wherein said separation unit is a methylthioalkyl moiety.

13. The method of claim 8, wherein said separation unit is a hydrocarbon chain introduced in a form of a linear or branched diol.

14. The method of claim 1, wherein a cleavable unit of either of said first or said second separation tags is selected from the group consisting of acid labile, fluoride ion labile, photolabile, redox labile, and electrophile labile moieties.

15. The method of claim 14, wherein said redox labile moiety comprises a dithioformacetal moiety.

16. The method of claim 1, wherein a cleavable unit of either of said first or said second separation tags comprises a siloxyl or disyloxyl moiety.

17. The method of claim 1, wherein said separation medium is selected from the group consisting of affinity, hydrophobic interaction, hydrophilic interaction, metal-chelating, ion exchange, covalent coupling, and antigen-antibody affinity separation media.

18. The method of claim 1, wherein said separation medium is an ion exchange separation medium.

19. The method of claim 1, wherein said separation medium is a reversed phase separation medium.

20. The method of claim 1, wherein said separation medium is a mixed-mode type separation medium.

21. The method of claim 20, wherein said mixed-mode type separation medium comprises reversed phase and ion exchange separation media.

22. The method of claim 20, wherein said mixed-mode type separation medium comprises a covalent coupling separation medium.

23. The method of claim 22, wherein said covalent coupling separation medium is based on the formation of a disulfide bond.

24. The method of claim 1, wherein said method further comprises eluting said at least one bifunctional oligonucleotide.

25. The method of claim 1, wherein said separation medium comprises a first separation medium and a second separation medium, said first separation medium effective for adhering to said first separation tag and said second separation medium effective for adhering to said second separation tag.

26. The method of claim 1, said method further comprising:
d) cleaving either said first separation tag or said second separation tag; and
e) eluting an oligonucleotide lacking said non-cleaved separation tag.

27. A method for separating oligonucleotides, said method comprising:
a) providing a plurality of oligonucleotides, wherein said plurality of oligonucleotides comprises at least one bifunctional oligonucleotide and at least one non-bifunctional oligonucleotide, and wherein each said at least one bifunctional oligonucleotide comprises a first separation tag attached to a first end of said at least one bifunctional oligonucleotide and a second separation tag attached to a second end of said at least one bifunctional oligonucleotide, and wherein cleavage of said first or said second separation tags yields an oligonucleotide having a 3′ hydroxyl moiety;
b) contacting said plurality of oligonucleotides with a separation medium under conditions effective for adhering said at least one bifunctional oligonucleotide to said separation medium;
c) eluting non-bifunctional oligonucleotides lacking said first separation tag without eluting said bifunctional oligonucleotides;
d) cleaving said first separation tag from the oligonucleotides retained on the separation medium; and
e) eluting non-bifunctional oligonucleotides lacking said second separation function.

28. The method of claim 27, wherein said cleaving step is facilitated using TBAF.

29. The method of claim 27, wherein said cleaving step is facilitated using an acid.

30. A composition comprising:
a) a plurality of oligonucleotides, each said oligonucleotide comprising a first separation tag attached to a first end of said oligonucleotide and a second separation tag attached to a second end of said oligonucleotide, wherein cleavage of said first or said separation tags yields an oligonucleotide having a 3′ hydroxyl moiety; and
b) a separation medium, said plurality of oligonucleotides adhering to said separation medium.

31. The composition of claim 30, wherein said separation medium comprises a first separation medium and a second separation medium, said first separation medium and said second separation medium being different separation media.

32. The method of claim 1, wherein a cleavable unit of either of said first or said second separation tags comprises an alkylthiomethyl moiety.

33. The method of claim 1, wherein a cleavable unit of either of said first or said second separation tags comprises a hydrocarbyldithiomethyl moiety.
